(19) United States
(12) Patent Application Publication
(10) Pub. No.: US 2015/0342563 A1
(43) Pub. Date: Dec. 3, 2015

(54) ULTRASONIC DIAGNOSTIC APPARATUS

(71) Applicants: KABUSHIKI KAISHA TOSHIBA, Minato-ku (JP); Toshiba Medical Systems Corporation, Otawara-shi (JP)

(72) Inventor: Sayaka TAKAHASHI, Otawara (JP)

(73) Assignees: KABUSHIKI KAISHA TOSHIBA, Minato-ku (JP); Toshiba Medical Systems Corporation, Otawara-shi (JP)

(21) Appl. No.: 14/718,581

(22) Filed: May 21, 2015

(30) Foreign Application Priority Data
May 30, 2014 (JP) 2014-113070

(51) Int. Cl. A61B 8/00; A61B 5/00; A61B 8/08

(52) U.S. Cl. CPC A61B 8/4416; A61B 8/5207; A61B 8/463; A61B 8/468; A61B 8/5223; A61B 8/5261; A61B 5/0077; A61B 5/444; A61B 2560/0475

(57) ABSTRACT

An ultrasonic diagnostic apparatus according to an embodiment includes an ultrasonic image generating circuitry, an imaging circuitry, a body mark storage and a body mark selection circuitry. The ultrasonic image generating circuitry generates an ultrasonic image via a probe brought into contact with a surface of an object. The imaging circuitry repeatedly obtains an optical image concerning the object. The body mark storage stores data of a plurality of types of body marks. The body mark selection circuitry selects a body mark corresponding to an examination region area of the object from the plurality of types of body marks by performing image analysis on the optical image.

### CROSS-REFERENCE TO RELATED APPLICATIONS

This application is based upon and claims the benefit of priority from the prior Japanese Patent Application No. 2014-113070, filed May 30, 2014 the entire contents of which are incorporated herein by reference.

### FIELD

Embodiments described herein relate generally to an ultrasonic diagnostic apparatus.

### BACKGROUND

In an ultrasonic examination, an examiner sometimes simultaneously perform a medical inquiry, visual examination, and palpation in addition to image diagnosis by an ultrasonic diagnostic apparatus. Therefore, the ultrasonic diagnostic apparatus is required to be designed for easy and efficient operations. In case of emergency medical care, in particular, initial diagnosis using the ultrasonic diagnostic apparatus needs to be quick. The ultrasonic diagnostic apparatus is used in place of a stethoscope in case of emergency medical care because of its feature of allowing easy examination.

It is, however, difficult to determine, based on only the image, which region the image represents because of the nature of an ultrasonic diagnostic image. For this reason, ultrasonic images are often stored with body marks being added to them to allow discrimination of examination target regions. Although the examiner manually adds such body marks at or after an examination, it is cumbersome for the examiner to add body marks, resulting in a deterioration in examination efficiency.

### BRIEF DESCRIPTION OF THE DRAWING

FIG. 1 is a block diagram showing the arrangement of an ultrasonic diagnostic apparatus according to the first embodiment;

FIG. 2 is a view showing a procedure in which a registration unit in FIG. 1 stores a VF image and a display image in an ultrasonic image storage unit in association with an ultrasonic image;

FIG. 3 is a flowchart showing a typical procedure for a series of operations according to the first embodiment;

FIG. 4 is a flowchart showing a typical procedure for a VF image generation subroutine according to the first embodiment;

FIG. 5 is a view for explaining about recording of VF images in a proper range by a proper range image extraction unit in FIG. 1;

FIG. 6 is a flowchart showing a typical procedure for a display image generation subroutine according to the first embodiment;

FIG. 7 is a view for explaining about the generation of a display image by the display image generation unit in FIG. 1;

FIG. 8 is a block diagram showing the arrangement of an ultrasonic diagnostic system according to a modification of the first embodiment;

FIG. 9 is a block diagram showing an ultrasonic diagnostic apparatus according to the second embodiment;

FIG. 10 is a view schematically showing an operation example according to the second embodiment;

FIG. 11 is a flowchart showing a typical procedure for a series of operations according to the second embodiment;

FIG. 12 is a flowchart showing a typical procedure for a comment creation subroutine according to the second embodiment;

FIG. 13 is a view for explaining how speech is delimited in units of sentences by a speech conversion unit in FIG. 9;

FIG. 14 is a view for explaining how speech is delimited in units of character strings by the speech conversion unit in FIG. 9;

FIG. 15 is a view showing an LUT which associates keywords with standard comments according to the second embodiment;

FIG. 16 is a view showing an example of how a keyword conversion unit in FIG. 9 converts keywords into comments;

FIG. 17 is a block diagram showing the arrangement of an ultrasonic diagnostic apparatus according to a modification of the second embodiment;

FIG. 18 is a block diagram showing the arrangement of an ultrasonic diagnostic apparatus according to the third embodiment; and

FIG. 19 is a flowchart showing a typical procedure for a series of operations according to the third embodiment.

### DETAILED DESCRIPTION

An ultrasonic diagnostic apparatus according to an embodiment includes an ultrasonic image generating circuitry configured to generate an ultrasonic image via a probe brought into contact with a surface of an object, an imaging circuitry configured to repeatedly obtain an optical image concerning the object, a body mark storage configured to store the data of a plurality of types of body marks, and a body mark selection circuitry configured to select a body mark corresponding to an examination region area of the object from the plurality of types of body marks by performing image analysis on the optical image.

An ultrasonic diagnostic apparatus according to an embodiment will be described with reference to the accompanying drawing. Note that the same reference numerals in the following description denote constituent elements having almost the same functions and arrangements, and a repetitive description will be made only when required.

### First Embodiment

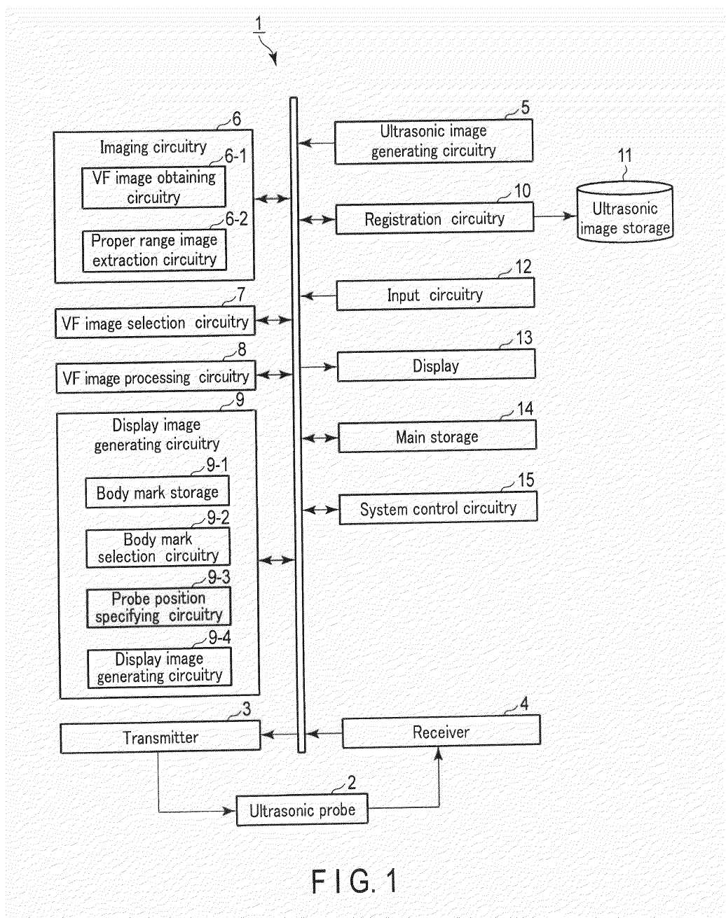

First of all, FIG. 1 is a block diagram showing the arrangement of an ultrasonic diagnostic apparatus 1 according to the first embodiment. The ultrasonic diagnostic apparatus 1 includes an ultrasonic probe 2, a transmitter 3, a receiver 4, an ultrasonic image generating circuitry 5, an imaging circuitry 6, a visual field (VF) image selection circuitry 7, a VF image processing circuitry 8, a display image generating circuitry 9, a registration circuitry 10, an ultrasonic image storage 11, an input circuitry 12, a display 13, a main storage 14, and a system control circuitry 15.

The ultrasonic probe 2 includes a plurality of transducers arrayed one-dimensionally or two-dimensionally. Each transducer generates an ultrasonic wave corresponding to a driving signal from the transmitter 3, and converts a reflected wave from an object into an electrical signal (echo signal). A matching layer is attached on the front side of a plurality of transducers to match the acoustic impedance differences between the transducers and the object. A backing member is attached on the rear side of the plurality of transducers to prevent the propagation of ultrasonic waves. When an ultrasonic wave is transmitted from the ultrasonic probe 2 to an object, the ultrasonic wave is sequentially reflected by a discontinuity surface of acoustic impedance of an internal body tissue. The reflected ultrasonic wave is received as an echo signal by the reception unit 4 via the ultrasonic probe. The amplitude of this echo signal depends on the acoustic impedance differences on the discontinuity surface by which the ultrasonic wave is reflected. When an ultrasonic wave is reflected by a blood flow, the surface of cardiac wall, or the like, the echo signal is subjected to a frequency shift depending on the velocity component of the moving body in the ultrasonic transmission direction due to the Doppler Effect.

The transmitter 3 includes a trigger generation circuit, delay circuit, and pulser circuit (none of which are shown). The pulser circuit repeatedly generates rate pulses for the formation of transmission ultrasonic waves at a predetermined rate frequency fr [Hz] (period: 1/fr sec). The delay circuit gives each rate pulse a delay time corresponding to a transmission direction and a transmission focal position for each channel. The trigger generating circuit applies a driving signal to the ultrasonic probe 2 at the timing based on this rate pulse. Upon application of a driving signal, the ultrasonic probe 2 transmits an ultrasonic transmission beam concerning the transmission direction and transmission focal position corresponding to a delay time.

The receiver 4 includes an amplifier circuit, A/D converter, and beam former (none of which are shown). The amplifier circuit amplifies an echo signal from the ultrasonic probe 2 for each channel. The A/D converter A/D-converts each amplified echo signal. The beam former gives each digital echo signal a delay time necessary to determine the beam direction of an ultrasonic reception beam for each reception focal position, and adds the echo signals to which the delay times have been given. This delay addition generates a reception signal corresponding to the ultrasonic reception beam.

The ultrasonic image generating circuitry 5 generates the data of an ultrasonic image corresponding to a video mode based on a reception signal. If the video mode is the B mode, the ultrasonic image generating circuitry 5 generates a B-mode image based on B-mode data. If the video mode is the Doppler mode, the ultrasonic image generating circuitry 5 generates a Doppler image representing a Doppler waveform based on Doppler data.

The imaging circuitry 6 includes a visual field image obtaining circuitry 6-1 and a proper range image extraction circuitry 6-2.

The VF image obtaining circuitry 6-1 is implemented by, for example, an optical camera. The optical camera is, for example, a wearable optical camera. The optical camera repeatedly obtains an optical image (to be referred to as a VF image hereafter) with respect to a range approximated to an examiner's visual field. In addition, the optical camera may be installed on a camera stand or a wall or ceiling of an examination room. Note that the VF image obtaining circuitry 6-1 may be implemented by an infrared camera. The VF image obtaining circuitry 6-1 stores VF images corresponding to the storage capacity (buffer area) of a built-in camera. More specifically, the VF image obtaining circuitry 6-1 stores obtained VF images in real time in the FIFO (First In First Out) format in accordance with the buffer area. More specifically, if there is no free space in the storage capacity of the VF image obtaining circuitry 6-1, the oldest VF image is deleted, and the newest VF image is recorded. The buffer area is limited by, for example, the resolution of each VF image and the hardware configuration. The respective VF images are recorded in association with the generation times in chronological order.

The proper range image extraction circuitry 6-2 records VF images, of the VF images repeatedly obtained by the VF image obtaining circuitry 6-1, which fall within a proper range. Upon execution of an ultrasonic imaging operation, the proper range image extraction circuitry 6-2 extracts an image in a proper range from the VF images obtained by the VF image obtaining circuitry 6-1 going back from this time point. A proper range will be described in detail later.

Note that the imaging circuitry 6 may be provided separately from the ultrasonic diagnostic apparatus 1. In this case, the imaging circuitry 6 and the ultrasonic diagnostic apparatus 1 may be connected to each other wiredly or wirelessly.

The VF image selection circuitry 7 selects a proper VF image from VF images in a proper range. More specifically, the VF image selection circuitry 7 selects a proper VF image from a plurality of VF images (VF images corresponding to the time of a proper range) which almost match in the imaging time of an ultrasonic image, based on how the contours of an image area corresponding to the ultrasonic probe 2 and an examination region area of the patient are depicted. Selection of a proper VF image will be described in detail later.

The VF image processing circuitry 8 performs image processing such as enlargement/reduction, moving, and trimming for a selected VF image to make the selected VF image have a proper size and be located at a proper position as a VF image. Note that the VF image processing circuitry 8 corrects even a VF image obtained at a distance from the examiner's visual field to have a proper size and be located at a proper position as a VF image as much as possible by performing image processing for the image. The VF image processing circuitry 8 corrects even a VF image in which a portion of an object or a portion of the ultrasonic probe 2 is reflected, by performing image processing, into a VF image having a proper size and located at a proper position as much as possible.

The display image generating circuitry 9 includes a body mark storage 9-1, a body mark selection circuitry 9-2, a probe position specifying storage 9-3, and a display image generating circuitry 9-4.

The body mark storage 9-1 stores image patterns of a plurality of types of body marks for each examination.

The body mark selection circuitry 9-2 selects a body mark corresponding to an examination region area of an object from a plurality of types of body marks by performing image analysis on a VF image. Image analysis is, for example, contour extraction. Note that the body mark selection circuitry 9-2 may select a body mark based on a VF image obtained at almost the same time as when the input circuitry 12 has received an input. The body mark selection circuitry 9-2 will be described in detail later.

The probe position specifying circuitry 9-3 specifies the position of the ultrasonic probe 2 at the selected body mark based on the selected VF image. More specifically, the probe position specifying circuitry 9-3 specifies the distal end position of the ultrasonic probe 2 by contour extraction from the VF image selected by the VF image selection circuitry 7. Note that contour extraction may be implemented by image recognition using a marker added to a predetermined position of a contour extraction filter or ultrasonic probe 2 or by other methods.

The display image generating circuitry 9-4 generates a display image concerning the selected body mark, to which a probe mark indicating the specified position is added. In other words, the display image generating circuitry 9-4 adds probe marks, each indicating the position of the probe corresponding to each of a plurality of contact positions of the probe on an object, to a body mark representing an examination region of the object.

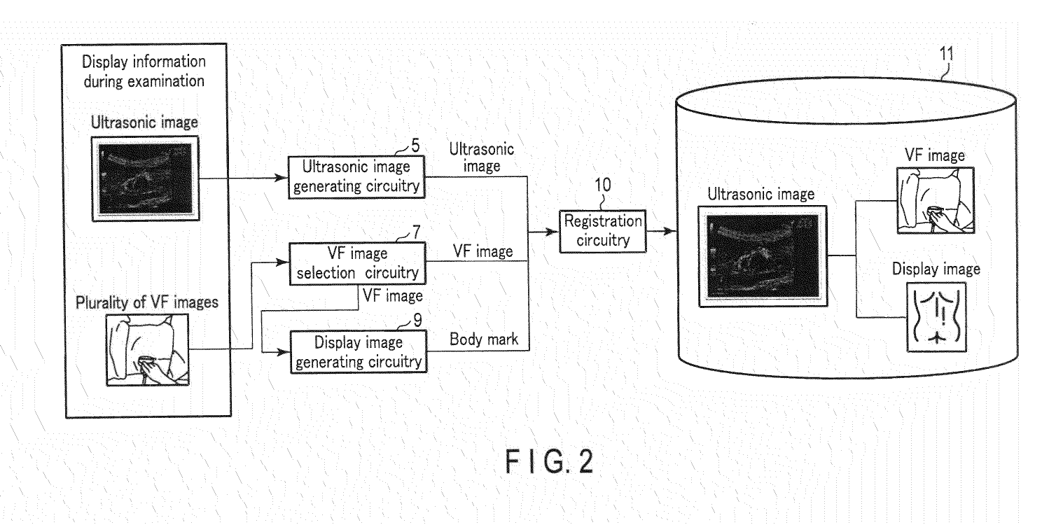

The registration circuitry 10 stores, in the ultrasonic image storage 11, the VF image selected by the VF image selection circuitry 7 and the display image in association with the ultrasonic image generated by the ultrasonic image generating circuitry 5. FIG. 2 is a view showing a procedure in which the registration circuitry 10 in FIG. 1 stores, in the ultrasonic image storage 11, a VF image and a display image in association with an ultrasonic image.

The ultrasonic image storage 11 stores an ultrasonic image. More specifically, the ultrasonic image storage unit 11 stores, for example, a selected body mark, a VF image used for the selection of the body mark, and the specified position of the ultrasonic probe 2 in association with an ultrasonic image. Note that the ultrasonic image storage 11 may store an ultrasonic image based on an input from the operator to the input circuitry 12. The ultrasonic image storage 11 may store the ultrasonic image generated by the ultrasonic image generating circuitry 5 in accordance with the instruction input by the operator in advance to the input circuitry 12. Note that the ultrasonic image storage 11 may store a selected VF image and information other than a selected body mark in association with an ultrasonic image.

The input circuitry 12 generates operation signals representing various types of instructions, commands, information, selections, settings, and the like desired by the examiner. The input circuitry 12 inputs the generated various types of instructions, commands, information, selections, settings, and the like to the system control circuitry 15. More specifically, for example, the input circuitry 12 accepts an operator's instruction to store an ultrasonic image. Note that the input circuitry 12 may include a memory. The memory stores, for example, the time when the operator's instruction to store an ultrasonic image is received.

The display 13 displays the ultrasonic image generated by the ultrasonic image generating circuitry 5, the VF image obtained by the imaging circuitry 6, a body mark, a display image, and the like. The display 13 displays various types of information.

The main storage 14 stores various types of data. The main storage 14 stores various types of predetermined values and various types of thresholds.

The system control circuitry 15 functions as the main unit of the ultrasonic diagnostic apparatus 1. The system control circuitry 15 implements various types of operations according to the first embodiment by comprehensively controlling the respective constituent elements included in the ultrasonic diagnostic apparatus 1.

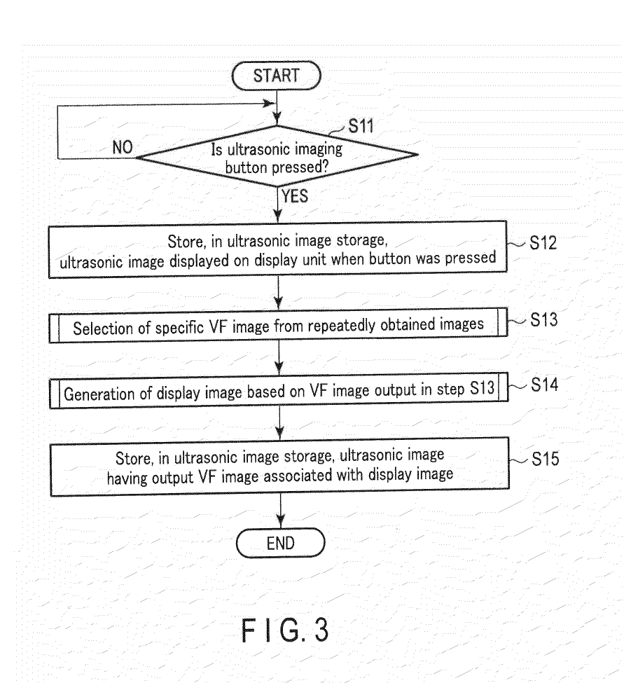

An operation example of the ultrasonic diagnostic apparatus 1 according to the first embodiment will be described in detail below. An example of a series of operations according to the embodiment will be described with reference to FIG. 3. FIG. 3 is a typical procedure for a series of operations according to the first embodiment. Assume that at the start of step S11, an object has been repeatedly scanned with ultrasonic waves via the ultrasonic probe 2, and the ultrasonic image generating circuitry 5 has generated ultrasonic images. The display 13 displays the repeatedly generated ultrasonic images in the form of a moving image. Assume also that VF images have been repeatedly obtained.

First of all, the system control circuitry 15 waits until an ultrasonic imaging button is pressed in accordance with an instruction from the examiner (step S11). The system control circuitry 15 keeps waiting until the ultrasonic imaging button is pressed.

When the ultrasonic imaging button is pressed, the system control circuitry 15 causes the registration circuitry 10 to store, in the ultrasonic image storage 11, the ultrasonic image displayed on the display 13 when the ultrasonic imaging button is pressed (step S12).

Upon execution of step S12, the system control circuitry 15 causes the VF image selection circuitry 7 to select a VF image (step S13). In step S13, the VF image selection circuitry 7 selects a specific VF image from the plurality of VF images obtained by the imaging circuitry 6.

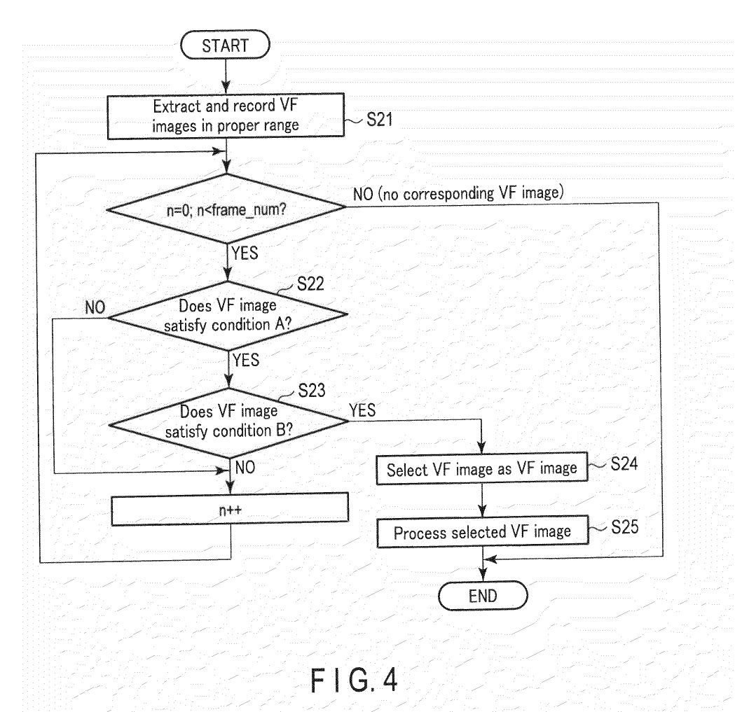

An operation example in a VF image generation subroutine in this embodiment will be described below with reference to FIG. 4. FIG. 4 is a flowchart showing a typical procedure for the VF image generation subroutine according to the first embodiment.

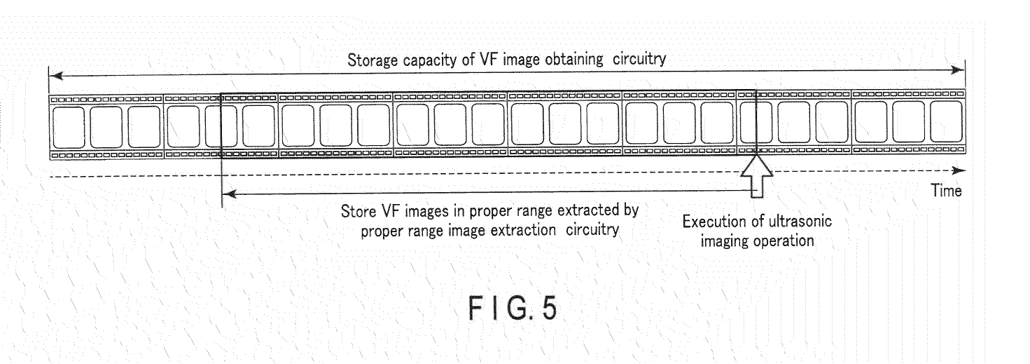

First of all, the system control circuitry 15 causes the proper range image extraction circuitry 6-2 to extract and record VF images in a proper range (step S21). FIG. 5 is a view for explaining about the recording of VF images in the proper range by the proper range image extraction circuitry 6-2 in FIG. 1. In this case, a proper range is defined as the range of a plurality of VF images in which the positional relationship between the ultrasonic probe 2 and the patient is almost the same as that at the time of execution of an imaging operation. More specifically, the VF images in the proper range are 13 VF images going back from the time of the execution of the ultrasonic imaging operation in FIG. 5. VF images in a proper range are set as consecutive VF images which satisfy the following conditions. When an acceleration sensor is provided for the ultrasonic probe 2, the proper range image extraction circuitry 6-2 calculates the amount of change (movement amount) from the position of the ultrasonic probe 2 at the time of the execution of an imaging operation by using an output from the acceleration sensor. The proper range image extraction circuitry 6-2 stores, as VF images in the proper range, consecutive VF images corresponding to the calculated amounts of change which fall within a predetermined range. Assume, however, that the number of consecutive VF images does not exceed a predetermined maximum number of images recorded. Note that the proper range image extraction circuitry 6-2 may store only a predetermined number of visual field images going back from the time of the execution of an ultrasonic imaging operation without using the acceleration sensor like in the above case. The predetermined number of images are stored in advance in a built-in memory.

Upon execution of step S21, the system control circuitry 15 causes the VF image selection circuitry 7 to determine whether a VF image as a processing target includes a probe area (step S22). In step S22, the VF image selection circuitry 7 determines whether the probe area is included. The VF image selection circuitry 7 determines, in reverse chronological order of VF images, whether each VF image includes the probe area. The built-in memory stores a plurality of probe image patterns. The VF image selection circuitry 7 extracts the contour of the ultrasonic probe 2 from the images in the proper range which are recorded by the proper range image extraction circuitry 6-2. If the matching ratio between the extracted contour of the ultrasonic probe 2 and one of the probe image patterns is equal to or more than a threshold, it is determined that the ultrasonic probe 2 can be discriminated. If it is determined in step S22 that the VF image does not include the probe area (NO in step S22), the system control circuitry 15 causes the VF image selection circuitry 7 to execute step S22 with respect to the next VF image. Note that VF images may be processed in a different order instead of reverse chronological order.

If it is determined in step S22 that the VF image includes the probe area (YES in step S22), the system control circuitry 15 causes the VF image selection circuitry 7 to determine whether the VF image includes a region area of the patient's body (step S23). In this case, a region of a patient's body is defined as an examination target region. The body mark storage 9-1 stores image patterns of a plurality of types of body marks. The VF image selection circuitry 7 extracts a contour area of the patient's body from the VF images in the proper range. If the matching ratio between the extracted contour of the patient's body and one of the image patterns of the body marks is equal to or more than the threshold, it is determined that the region of the patient's body can be discriminated.

If it is determined in step S23 that the VF image does not include the region area of the patient's body (NO in step S23), the system control circuitry 15 causes the VF image selection circuitry 7 to execute step S22 with respect to a VF image as the next processing target. Note that if it is determined in steps S22 and S23 that there is no VF image including both the probe area and the region area of the patient's body, the VF image selection circuitry 7 determines that there is no proper VF image. The system control circuitry 15 terminates the VF image generation subroutine.

If it is determined in step S23 that the VF image includes the region area of the patient's body (YES in step S23), the system control circuitry 15 causes the VF image selection circuitry 7 to select the VF image as a visual field image (step S24).

Upon execution of step S24, the system control circuitry 15 causes the VF image processing circuitry 8 to process the selected visual field image (step S25). In step S25, the VF image processing circuitry 8 processes the selected visual field image into an image having a proper size and located at a proper position by enlargement/reduction, moving, trimming, and the like. Note that step S25 may be omitted.

The description will return to the operation example of the ultrasonic diagnostic apparatus 1 from the description of the visual field image generation subroutine.

Upon execution of step S13, the system control unit 15 causes the display image generating circuitry 9 to generate a display image based on the VF image output in step S13 (step S14).

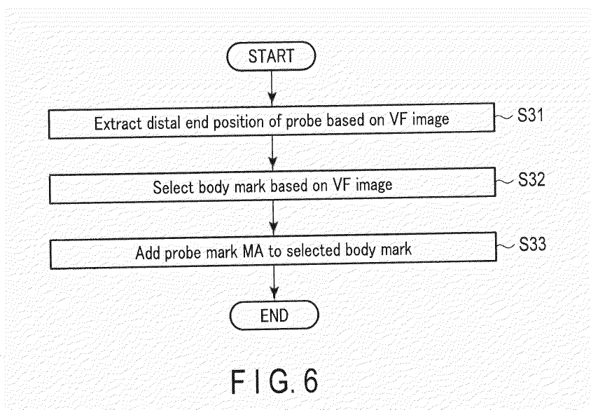

A display image generation subroutine according to the first embodiment will be described in detail below. An example of a series of operations according to this embodiment will be described with reference to FIG. 6. FIG. 6 is a flowchart showing a typical procedure for the display image generation subroutine according to the first embodiment.

First of all, the system control circuitry 15 causes the probe position specifying circuitry 9-3 to perform position specifying processing (step S31). In step S31, the probe position specifying circuitry 9-3 specifies the distal end position of the ultrasonic probe 2 by processing the VF image selected by the VF image generation subroutine. More specifically, for example, the probe position specifying circuitry 9-3 specifies the distal end position of the ultrasonic probe 2 by performing contour extraction processing for the selected VF image.

Upon execution of step S31, the system control circuitry 15 causes the body mark selection circuitry 9-2 to perform selection processing (step S32). In step S32, the body mark selection circuitry 9-2 performs body mark selection processing by processing the selected VF image. More specifically, for example, the body mark selection circuitry 9-2 performs contour extraction processing for the selected VF image area. The body mark selection circuitry 9-2 compares the region of the patient's body having undergone contour extraction with the image patterns of a plurality of types of body marks stored in the body mark storage 9-1 to select the image pattern of a body mark which exhibits the highest similarity.

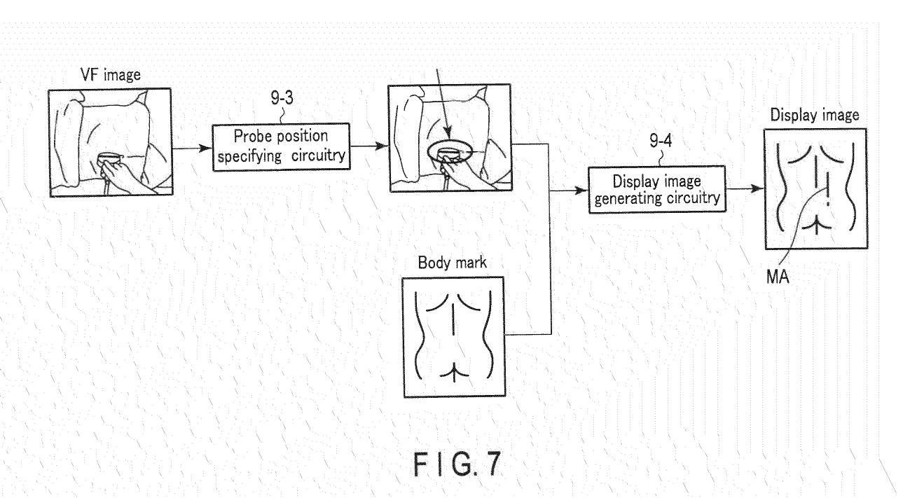

Upon execution of step S32, the system control unit 15 causes the display image generating circuitry 9-4 to add a probe mark MA indicating the distal end position of the ultrasonic probe 2 specified in step S31 to the body mark selected in step S32 upon position matching (step S33). FIG. 7 is a view for explaining about the generation of a display image by the display image generating circuitry 9-4 in FIG. 1. The display image generating circuitry 9-4 adds the probe mark MA indicating the distal end position of the ultrasonic probe 2, which is specified by the probe position specifying circuitry 9-3, to the body mark selected by the body mark selection circuitry 9-2. A body mark to which a mark indicating the distal end position of the ultrasonic probe 2 is added will be referred to as a display image hereinafter.

The description will return to the operation example of the ultrasonic diagnostic apparatus 1 from the description of the display image generation subroutine.

Upon execution of step S14, the system control unit 15 causes the registration circuitry 10 to store, in the ultrasonic image storage 11, an ultrasonic image having an output VF image associated with a display image (step S15).

With that, the description of the operation example according to the first embodiment is finished.

As described above, based on a VF image, the ultrasonic diagnostic apparatus 1 according to the first embodiment can automatically select a body mark corresponding to the VF image. In addition, based on a VF image, the ultrasonic diagnostic apparatus 1 can automatically select a probe mark indicating a probe position corresponding to the VF image. The ultrasonic image storage 11 can store a VF image and a body mark to which a probe mark indicating a probe position is added in association with an ultrasonic image. Therefore, the examiner can perform an ultrasonic examination without manually adding any body mark during the examination. The ultrasonic diagnostic apparatus 1 according to this embodiment can therefore improve the examination efficiency in ultrasonic examination.

### Modification of First Embodiment

The above embodiment has exemplified the ultrasonic diagnostic apparatus 1. However, in the above embodiment, the ultrasonic diagnostic apparatus 1 need not incorporate all the constituent elements, and, for example, processing by some constituent elements may be performed by a computer on a network. Processing by the computer on the network is implemented by, for example, cloud computing. Note that the same reference numerals in the following description denote constituent elements having almost the same functions and arrangements, and a repetitive description will be made only when required.

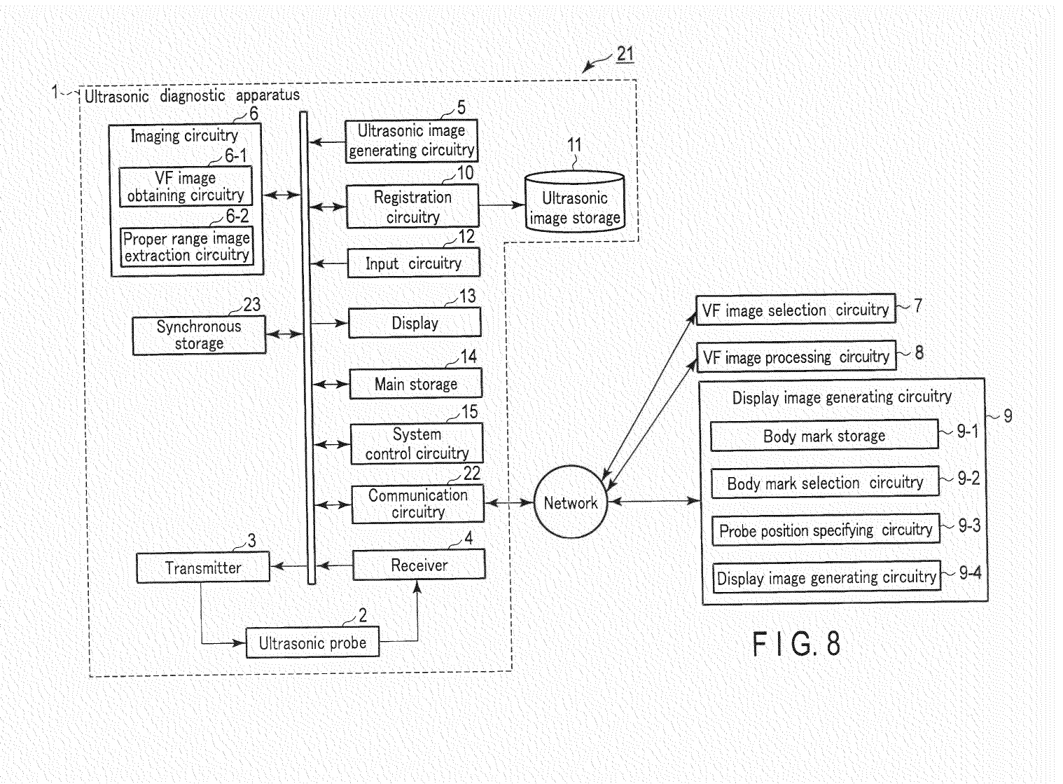

FIG. 8 is a block diagram showing the arrangement of an ultrasonic diagnostic apparatus 21 according to a modification of the first embodiment. The ultrasonic diagnostic apparatus 21 according to this modification includes a communication circuitry 22 and a synchronous storage 23 unlike the ultrasonic diagnostic apparatus 1 according to the above embodiment. In addition, in the ultrasonic diagnostic apparatus 21 according to the modification, the VF image selection circuitry 7, the VF image processing circuitry 8, and the display image generating circuitry 9 are implemented by a computer connected to the ultrasonic diagnostic apparatus 1 via a network.

The communication circuitry 22 communicates with the VF image selection circuitry 7, the VF image processing circuitry 8, and the display image generating circuitry 9 via the network. More specifically, the communication circuitry 22 transmits a plurality of VF images obtained by the imaging circuitry 6 to the VF image selection circuitry 7, the VF image processing circuitry 8, and the display image generating circuitry 9 on the network. The communication circuitry 22 transmits various instructions input via the input circuitry 12 to the VF image selection circuitry 7, the VF image processing circuitry 8, and the display image generating circuitry 9. Note that the communication circuitry 22 may also transmit information about the ultrasonic diagnostic apparatus 1, other than the above information, to the VF image selection circuitry 7, the VF image processing circuitry 8, and the display image generating circuitry 9.

The synchronous storage 23 stores the VF image selected by the VF image selection circuitry 7 on the network in synchronism with the selection of the image. The synchronous storage 23 stores the display image generated by the display image generating circuitry 9 on the network in synchronism with the generation of the image. The VF images and display images synchronously stored are sequentially stored in the ultrasonic image storage 11.

The ultrasonic diagnostic apparatus 21 according to this modification can implement VF image generation processing and display image generation processing by cloud computing. Therefore, the ultrasonic diagnostic apparatus 21 needs to incorporate only minimum necessary constituent elements. This reduces the processing performed by the ultrasonic diagnostic apparatus 21. The ultrasonic diagnostic apparatus 21 according to this embodiment can therefore improve the examination efficiency in ultrasonic examination.

### Second Embodiment

When making a medical inquiry, visual examination, and palpation during an ultrasonic examination, a recorder is required to record results of the medical inquiry and the like in addition to the examiner. Alternatively, the examiner records the results afterward. In any case, this is a cumbersome operation. In addition, when continuously diagnosing a plurality of patients or recording results of a medical inquiry, visual examination, and palpation after an examination, there is a risk of confusing patients. According to the second embodiment, therefore, speech is recorded at the time of a medical inquiry, visual examination, and palpation, and the character strings of the recorded speech are stored as results of the medical inquiry and the like in real time in association with an ultrasonic image. Note that the same reference numerals in the following description denote constituent elements having almost the same functions and arrangements, and a repetitive description will be made only when required.

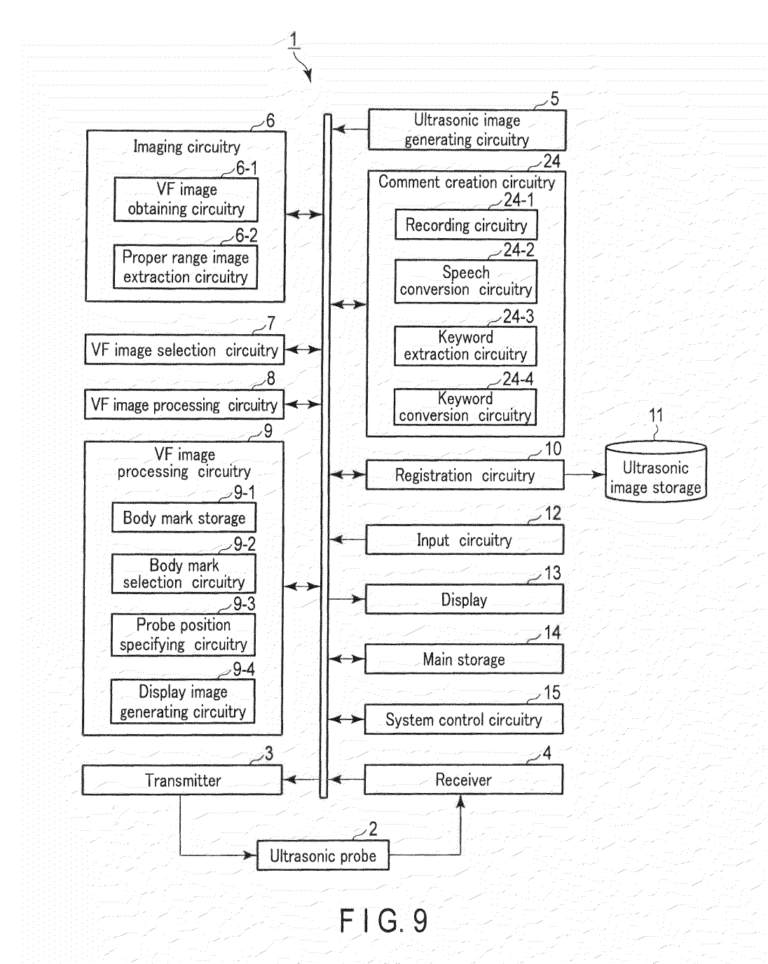

FIG. 9 is a block diagram showing the arrangement of an ultrasonic diagnostic apparatus 1 according to the second embodiment. The ultrasonic diagnostic apparatus 1 according to the second embodiment includes a comment creation circuitry 24 in addition to the constituent elements of the ultrasonic diagnostic apparatus 1 according to the first embodiment. The ultrasonic diagnostic apparatus 1 according to the second embodiment can store the comments created by the comment creation circuitry 24 in an ultrasonic image storage 11 in association with an ultrasonic image.

The comment creation circuitry 24 includes a recording circuitry 24-1, a speech conversion circuitry 24-2, a keyword extraction circuitry 24-3, and a keyword conversion circuitry 24-4.

The recording circuitry 24-1 records speech. The recording circuitry 24-1 is implemented by a microphone 24-1 and the like. More specifically, the microphone 24-1 receives conversations between a doctor or the like and a patient at the time of a medical inquiry, visual examination, and palpation via the diaphragm provided on the microphone 24-1, and converts the vibrations originating from the conversations into analog electrical signals.

The speech conversion circuitry 24-2 converts the recorded speech into character strings. The speech conversion circuitry 24-2 delimits the character strings in linguistic units. A linguistic unit is, for example, a sentence unit. Speech converted into a character string will be referred to as a speech text.

The keyword extraction circuitry 24-3 extracts preset keywords from a plurality of converted character strings. The preset keywords are stored in a built-in memory in advance. In this case, each keyword is set to a character string (word) representing the symptom of an object.

The keyword conversion circuitry 24-4 converts an extracted keyword into a standard comment associated with the extracted keyword in advance. Keyword conversion will be described in detail later.

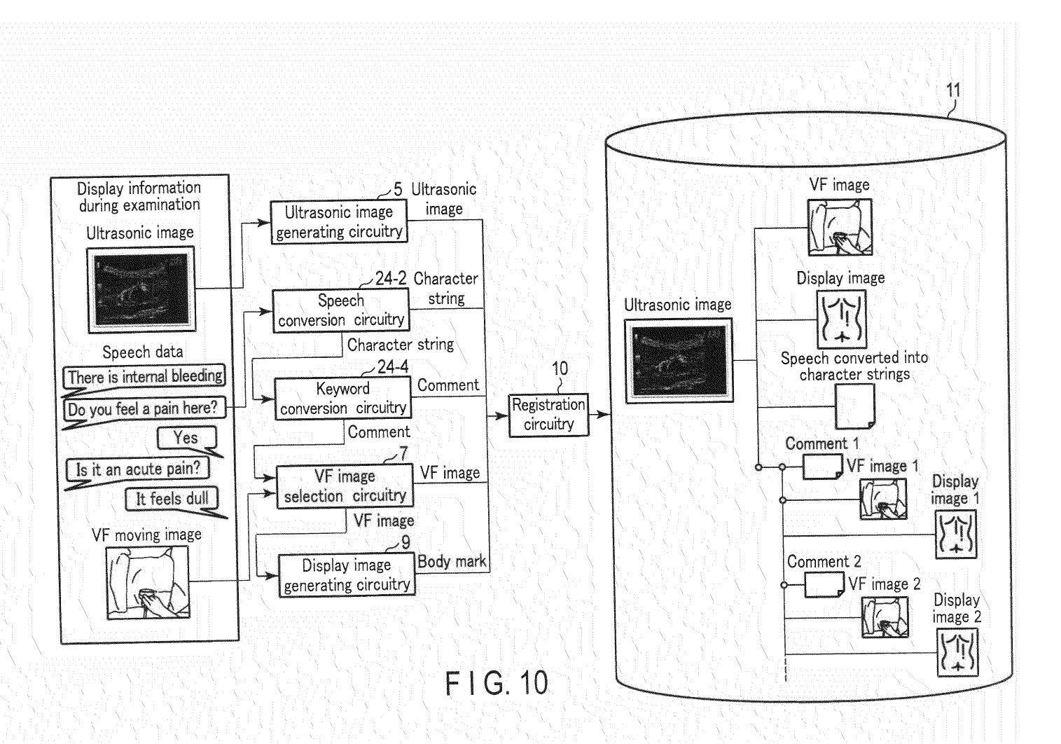

FIG. 10 is a view schematically showing an operation example according to the second embodiment. The registration circuitry 10 stores, for each comment, in the ultrasonic image storage 11, a visual field image corresponding to the comment and a body mark corresponding to the comment in association with each other. A speech text is divided according to a plurality of linguistic units and converted into standard comments. In this case, each standard comment is a sentence representing a symptom corresponding to a keyword. For example, a standard comment is written in a writing form allowing easy use for the creation of a report. In addition, each standard comment is a sentence which facilitates organization and browsing of symptom information. The registration circuitry 10 stores, in the ultrasonic image storage 11, standard comments, VF images corresponding to the comments, and body marks corresponding to the comments in association with an ultrasonic image.

An operation example of the ultrasonic diagnostic apparatus 1 according to the second embodiment will be described in detail below. An example of a series of operations in this embodiment will be described with reference to FIG. 11. FIG. 11 is a flowchart showing a typical procedure for a series of operations according to the second embodiment. Assume that at the start of step S41, an object has been repeatedly scanned with ultrasonic waves via an ultrasonic probe 2, and an ultrasonic image generation unit 5 has generated ultrasonic images. A display 13 displays the repeatedly generated ultrasonic images in the form of a moving image. Assume also that a VF image obtaining circuitry 6-1 has repeatedly obtained VF images. In addition, assume that the recording circuitry 24-1 has recorded speech. The recording circuitry 24-1 records conversations between a doctor or the like and a patient at the time of a medical inquiry, visual examination, and palpation.

First of all, a system control circuitry 15 waits until ultrasonic imaging operation input is performed in accordance with an instruction from the examiner (step S41). The system control circuitry 15 keeps waiting until the ultrasonic imaging button is pressed.

When an ultrasonic imaging operation is executed, the system control circuitry 15 causes the ultrasonic image generating circuitry 5 to generate an ultrasonic image from the signal received via the ultrasonic probe 2 (step S42). In step S42, the ultrasonic image generating circuitry 5 generates an ultrasonic image at the time point when the button is pressed. Note that in the moving image obtaining mode, the ultrasonic diagnostic apparatus 1 generates ultrasonic images corresponding to a predetermined past time from the time point when the button is pressed.

Upon execution of step S42, the system control circuitry 15 causes a VF image selection circuitry 7 to select a VF image (step S43). In step S43, the VF image selection circuitry 7 selects a VF image from the plurality of VF images obtained by an imaging circuitry 6. The VF image generation subroutine in step S43 is the same as that in the first embodiment.

Upon execution of step S43, the system control circuitry 15 causes a display image generating circuitry 9 to generate a display image based on the VF image generated in step S43 (step S44). The display image generation subroutine in step S44 is the same as that in the first embodiment.

Upon execution of step S44, the system control circuitry 15 causes the comment creation circuitry 24 to create standard comments based on speech (step S45).

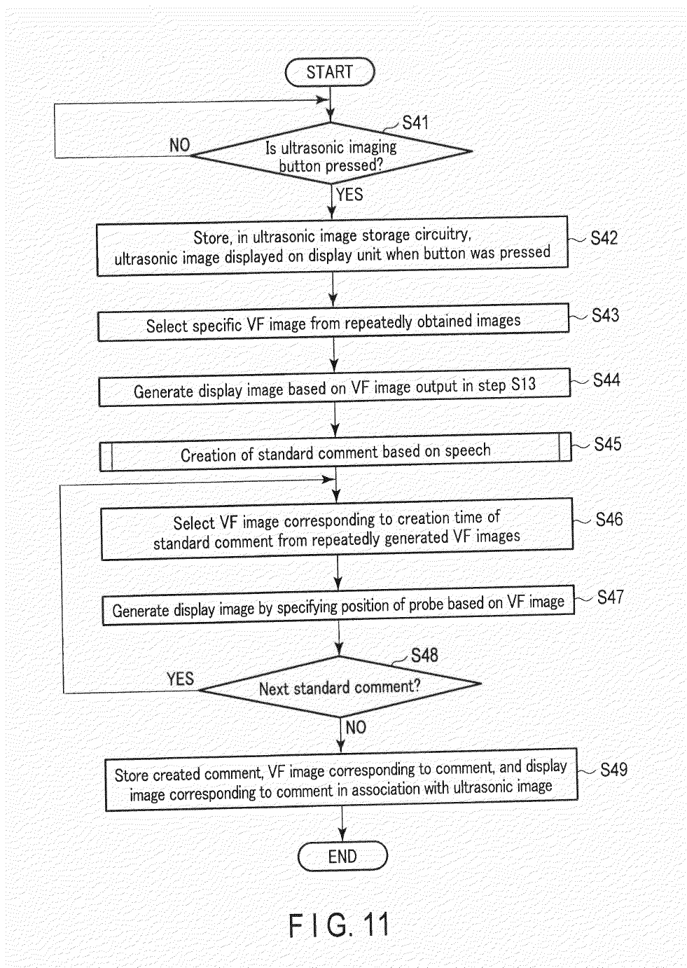

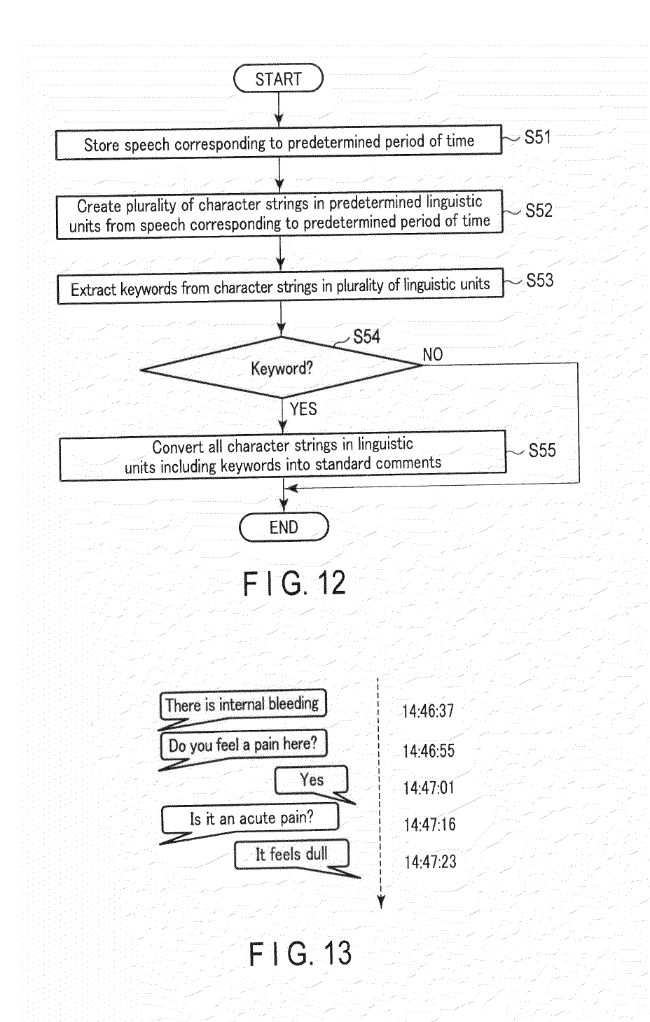

An operation example in a comment creation subroutine according to this embodiment will be described below with reference to FIG. 12. FIG. 12 is a flowchart showing a typical procedure for the comment creation subroutine according to the second embodiment.

Upon execution of step S44, the system control circuitry 15 causes the recording circuitry 24-1 to store speech corresponding to a predetermined time (step S51).

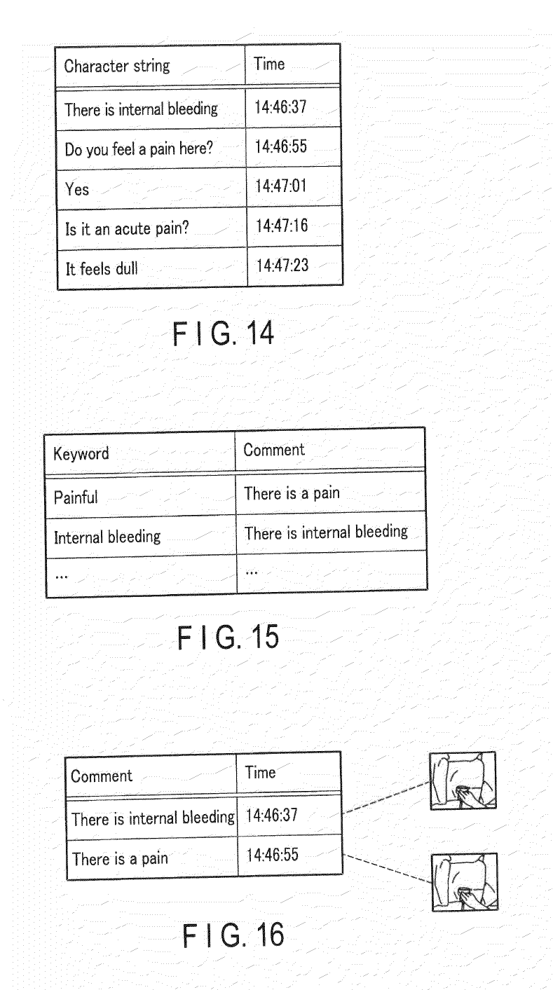

Upon execution of step S51, the system control circuitry 15 causes the speech conversion circuitry 24-2 to create a plurality of character strings in predetermined linguistic units from the speech corresponding to the predetermined time (step S52). In step S52, the speech conversion circuitry 24-2 delimits the speech corresponding to the predetermined time in predetermined linguistic units. A predetermined linguistic unit is, for example, a sentence unit. FIG. 13 is a view for explaining how the speech conversion circuitry 24-2 in FIG. 9 delimits speech in sentence units. FIG. 13 shows an example of a medical inquiry in case of emergency medical care. The speech conversion circuitry 24-2 records, on a built-in memory, each of utterances in sentence units in association with the times when the respective utterances were recorded. For example, FIG. 13 shows that the utterance “There is internal bleeding.” is stored in association with “14:46:37”. FIG. 14 is a view for explaining how the speech conversion circuitry 24-2 in FIG. 9 converts speech delimited in sentence units into character strings. For example, FIG. 14 shows that an utterance corresponding to the character string “There is internal bleeding.” is stored in association with “14:46:37”.

Upon execution of step S52, the system control circuitry 15 causes the keyword extraction circuitry 24-3 to extract keywords from character strings in a plurality of linguistic units (step S53). In step S53, the keyword extraction circuitry 24-3 determines whether any keyword is included in a plurality of delimited character strings. FIG. 15 is a view showing an LUT (Lookup Table) which associates keywords with standard comments according to the second embodiment. For example, FIG. 15 shows that when a given sentence includes the keyword “painful”, the sentence is converted into the standard comment “There is a pain.”

Upon execution of step S53, the system control circuitry 15 causes the keyword extraction circuitry 24-3 to determine whether a plurality of character strings include a keyword (step S54). If the keyword extraction circuitry 24-3 determines in step S54 that no keyword is included (NO in step S54), the comment creation subroutine is terminated.

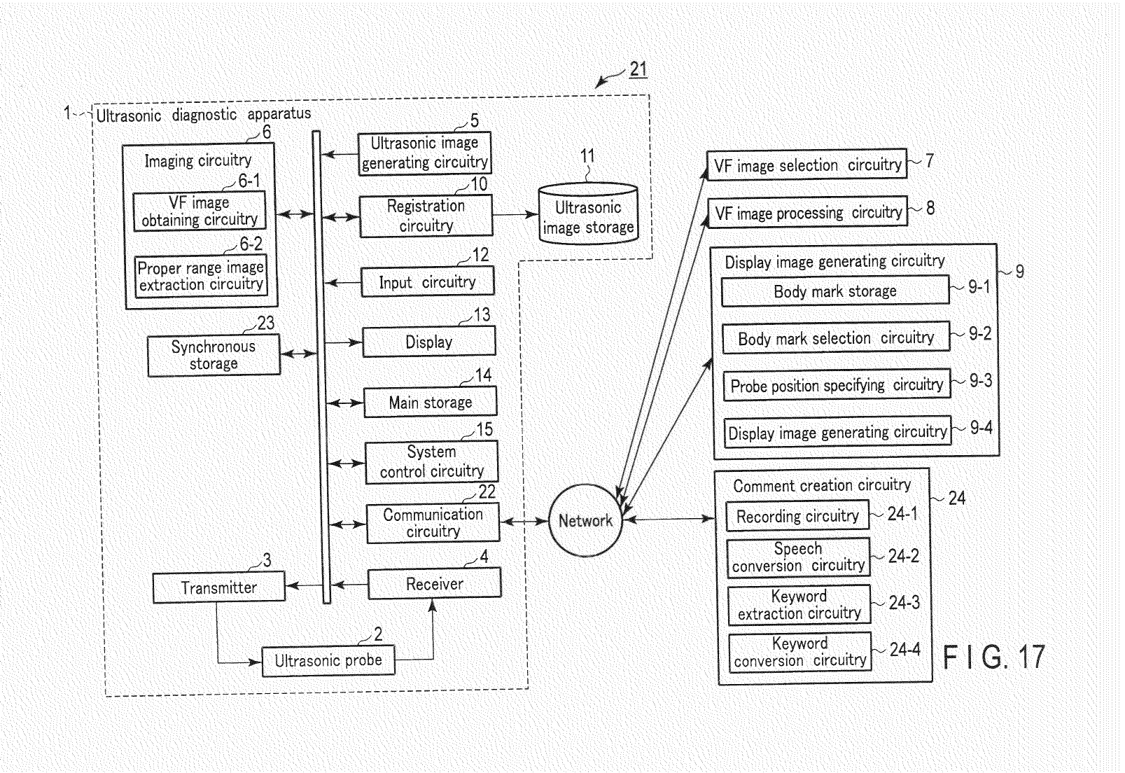

If the keyword extraction circuitry 24-3 determines in step S54 that a keyword is included (YES in step S54), the system control circuitry 15 causes the keyword conversion circuitry 24-4 to convert all the character strings in linguistic units including the keyword into standard comments (step S55). FIG. 16 is a view showing an example in which the keyword conversion circuitry 24-4 in FIG. 9 converts keywords into comments. In step S55, the keyword conversion circuitry 24-4 converts sentences including the keywords into standard comments corresponding to the keywords. The keyword conversion circuitry 24-4 stores the standard comments in the ultrasonic image storage unit 11 in association with the times corresponding to the comments.

The description of the operation example of the ultrasonic diagnostic apparatus 1 will be returned after the description of the comment creation subroutine.

Upon execution of step S45, the system control circuitry 15 causes the VF image selection circuitry 7 to select a VF image (step S46). The VF image selection circuitry 7 selects a VF image generated at almost the same time as that corresponding to the standard comment.

Upon execution of step S46, the system control circuitry 15 causes the display image generating circuitry 9 to specify the position of the probe based on the VF image generated in step S46 and generate a display image (step S47).

Upon execution of step S47, the system control circuitry 15 determines whether there is the next standard comment (step S48). If the system control circuitry 15 in step S48 that there is the next standard comment (YES in step S48), the process returns to step S46.

If the system control circuitry 15 determines in step S48 that there is no next standard comment (NO in step S48), the ultrasonic image storage 11 stores each created comment, a VF image corresponding to the comment, and a display image corresponding to the comment in association with an ultrasonic image (step S49).

With that, the description of the operation example according to the second embodiment is finished.

As described above, the ultrasonic diagnostic apparatus 1 according to the second embodiment can automatically store, in the ultrasonic image storage 11, a VF image, a display image to which a probe position is added, and a speech text in association with an ultrasonic image. The ultrasonic diagnostic apparatus 1 can further store, in the ultrasonic image storage 11, a plurality of standard comments and VF images and display images corresponding to the respective standard comments in association with each other. Therefore, the examiner need not manually write medical inquiry contents. In addition, storing speech by converting it into standard comments facilitates organization and browsing of symptom information as compared with a case in which speech is stored without any conversion. Furthermore, associating standard comments with VF images facilitates visually grasping the state of examination at the time of creation of a comment. Therefore, the ultrasonic diagnostic apparatus 1 according to this embodiment can improve the examination efficiency in an ultrasonic examination.

### Modification of Second Embodiment

The above embodiment has exemplified the ultrasonic diagnostic apparatus 1. However, in the above embodiment, all the constituent elements need not be incorporated in the ultrasonic diagnostic apparatus 1, and for example, processing by some constituent elements may be implemented by a computer on a network. The processing by the computer on the network is implemented by, for example, cloud computing. Note that the same reference numerals in the following description denote constituent elements having almost the same functions and arrangements, and a repetitive description will be made only when required.

FIG. 17 is a block diagram showing the arrangement of an ultrasonic diagnostic system 21 according to a modification of the second embodiment. The ultrasonic diagnostic system 21 according to this modification includes a communication circuitry 22 and a synchronous storage unit 23 unlike the ultrasonic diagnostic apparatus 1 according to the above embodiment. In the ultrasonic diagnostic system 21 according to the modification, the VF image selection circuitry 7, the VF image processing circuitry 8, and the display image generating circuitry 9 are implemented by a computer connected to the ultrasonic diagnostic apparatus 1 via a network.

The communication circuitry 22 communicates with the VF image selection circuitry 7, the VF image processing circuitry 8, and the display image generating circuitry 9 via the network. More specifically, the communication circuitry 22 transmits a plurality of VF images obtained by the imaging circuitry 6 to the visual field image selection unit 7, the VF image processing circuitry 8, and the display image generating circuitry 9 on the network. The communication circuitry 22 transmits various types of instructions input from an input circuitry 12 to the VF image selection circuitry 7, the VF image processing circuitry 8, and the display image generating circuitry 9. Note that the communication circuitry 22 may transmit information about the ultrasonic diagnostic apparatus 1, other than the above information, to the VF image selection circuitry 7, the VF image processing circuitry 8, and the display image generating circuitry 9.

In synchronism with the selection of the image, the synchronous storage 23 stores the VF image selected by the VF image selection circuitry 7 on the network. In addition, in synchronism with the generation of the image, the synchronous storage 23 stores the display image generated by the display image generating circuitry 9 on the network. The synchronously stored VF images and display images are sequentially stored in the ultrasonic image storage 11.

The ultrasonic diagnostic apparatus 1 according to this modification can implement VF image generation processing and display image generation processing by cloud computing. Therefore, the ultrasonic diagnostic apparatus 1 needs to incorporate only minimum necessary constituent elements. This reduces the processing performed by the ultrasonic diagnostic apparatus 1. The ultrasonic diagnostic apparatus 1 according to this embodiment can therefore improve the examination efficiency in ultrasonic examination.

### Third Embodiment

An ultrasonic diagnostic apparatus 1 according to the third embodiment will be described next.

Note that the same reference numerals in the following description denote constituent elements having almost the same functions and arrangements, and a repetitive description will be made only when required.

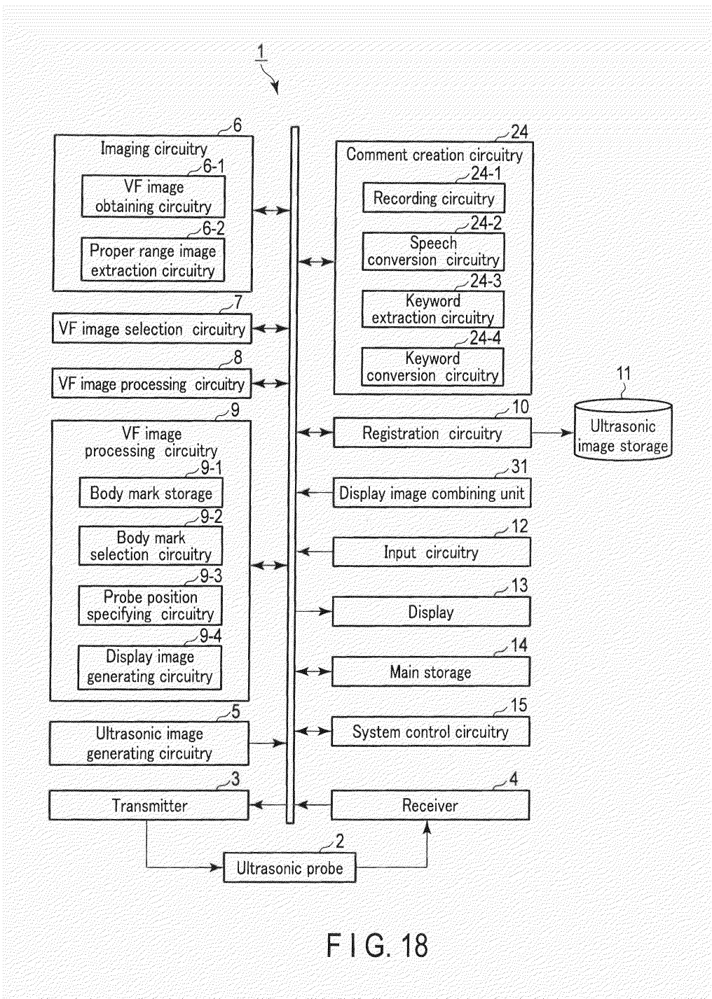

FIG. 18 is a block diagram showing the arrangement of the ultrasonic diagnostic apparatus 1 according to the third embodiment. The ultrasonic diagnostic apparatus 1 according to the third embodiment includes a display image combining circuitry 31 in addition to the respective types of constituent elements of the ultrasonic diagnostic apparatus 1 according to the second embodiment.

The display image combining circuitry 31 can combine a plurality of display images and store the resultant image in an ultrasonic image storage 11 in association with an ultrasonic image. In other words, the display image combining circuitry 31 generates a composite display image by adding probe marks corresponding to a plurality of contact positions of an ultrasonic probe 2 on an object to a body mark representing an examination region of the object.

The ultrasonic image storage 11 stores a composite display image, with symptom information representing the symptom of an object at each of a plurality of contact positions of the ultrasonic probe 2 being associated with each contact position. The ultrasonic image storage 11 stores the VF image selected by a VF image selection circuitry 7, a display image, and a speech text in association with the ultrasonic image generated by an ultrasonic image generating circuitry 5. The ultrasonic image storage 11 stores, for each comment, a VF image corresponding to the comment in association with a display image corresponding to the comment.

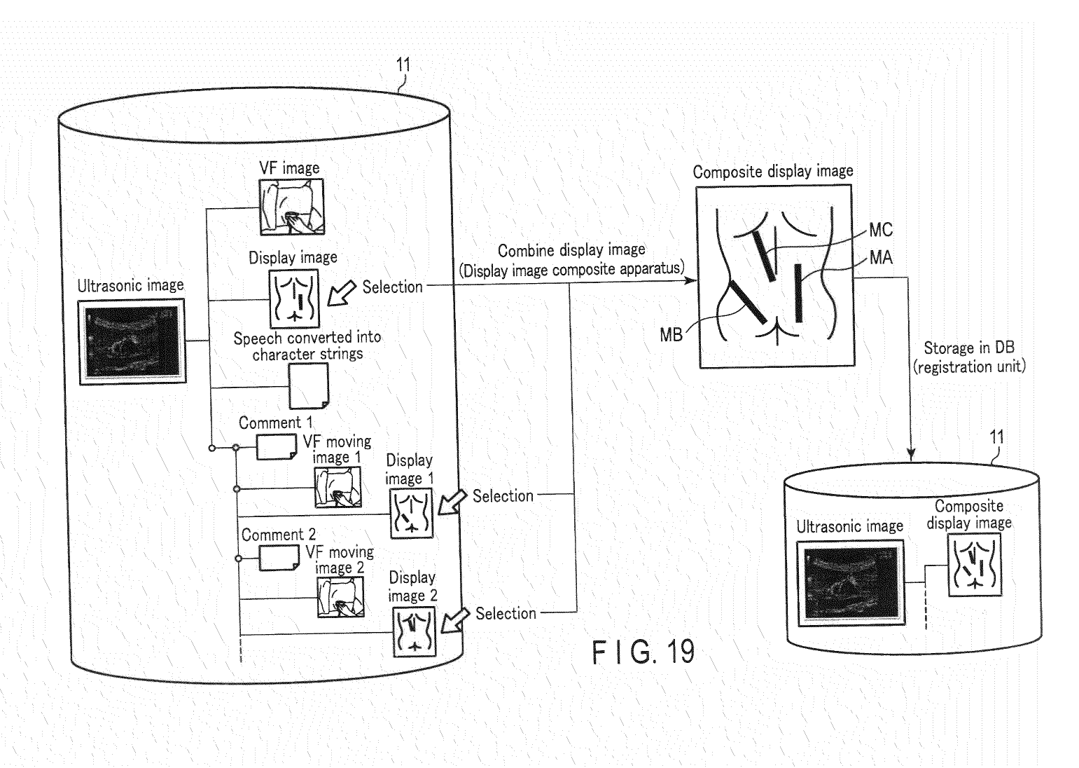

An operation example of the ultrasonic diagnostic apparatus 1 according to the third embodiment will be described below with reference to FIG. 19. FIG. 19 shows a typical procedure for a series of operations according to the third embodiment. The ultrasonic image storage 11 stores VF images, display images, and character strings in association with an ultrasonic image. In addition, VF images respectively corresponding to a plurality of comments and display images respectively corresponding to the comments are associated with the ultrasonic image. Each comment represents symptom information. Symptom information is information representing the symptom of an object at a contact position of the ultrasonic probe 2. For example, as shown in FIG. 19, VF image 1 corresponding to comment 1 and display image 1 corresponding to comment 1 are associated with comment 1. In this case, as shown in, for example, FIG. 16, comments include, for example, “There is internal bleeding.” and “There is a pain.” In addition, the display image combining circuitry 31 combines a display image (added with a probe mark MA) concerning an ultrasonic image, a display image (added with a probe mark MB) concerning comment 1, and a display image (added with a probe mark MC) concerning comment 2. The ultrasonic image storage 11 stores the composite display image. A plurality of probe marks are depicted on the composite display image.

A display 13 displays a composite display image. Each probe mark added to the display image is associated with a comment. When a probe mark is selected via an input circuitry 12, the display 13 displays the comment associated with the selected probe mark. If, for example, the probe mark MB is selected, comment 1 (e.g., “There is a pain.”) is displayed. Therefore, the examiner can check the symptom of the patient for each contact position.

The ultrasonic diagnostic apparatus 1 according to this embodiment allows the examiner to grasp symptoms at a plurality of positions from one composite display image. The ultrasonic diagnostic apparatus 1 according to the embodiment can therefore improve the examination efficiency in ultrasonic examination.

Although several embodiments have been described above, they are merely examples and not intended to limit the scope of the present invention. These novel embodiments can be implemented in other various forms, and various omissions, replacements, and changes can be made without departing from the spirit of the present invention. These embodiments and their modifications are incorporated in the scope and sprit of the present invention, and are also incorporated in the scope of the invention and its equivalents defined in the appended claims. For example, the following modifications are also incorporated in the scope of the present invention.

In addition, each function according to each embodiment can be implemented by installing a scattered radiation correction processing program in a computer such as a workstation and loading them into the memory. In this case, the programs which can cause the computer to execute the corresponding techniques can be distributed by being stored in storage media such as magnetic disks (floppy disks, hard disks, and the like), optical disks (CD-ROMs, DVDs, and the like), and semiconductor memories.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An ultrasonic diagnostic apparatus comprising:
an ultrasonic image generating circuitry configured to generate an ultrasonic image via a probe brought into contact with a surface of an object;
an imaging circuitry configured to repeatedly obtain an optical image concerning the object;
a body mark storage configured to store data of a plurality of types of body marks; and
a body mark selection circuitry configured to select a body mark corresponding to an examination region area of the object from the plurality of types of body marks by performing image analysis on the optical image.

2. The apparatus of claim 1, further comprising an optical image selection circuitry configured to select an optical image obtained at substantially the same time as an imaging time of the ultrasonic image from the repeatedly obtained optical images,
wherein the body mark circuitry unit selects a body mark similar to an object area corresponding to the object included in the selected optical image from the plurality of stored body marks.

3. The apparatus of claim 2, further comprising an ultrasonic image storage configured to store the selected optical image and the selected body mark in association with the ultrasonic image.

4. The apparatus of claim 2, further comprising:
a specifying circuitry configured to specify a position of the probe on the selected body mark based on the selected optical image; and
a display image generating circuitry configured to generate a display image concerning the selected body mark, to which the probe mark indicating the specified position is added.

5. The apparatus of claim 4, further comprising an ultrasonic image storage configured to store the selected body mark, an optical image used for the selection of the selected body mark, and the specified position in association with the ultrasonic image.

6. The apparatus of claim 5, further comprising:
a recording circuitry configured to record speech; and
a speech conversion circuitry configured to convert the recorded speech into character strings,
wherein the ultrasonic image storage further stores the converted character string in association with the ultrasonic image.

7. The apparatus of claim 6, further comprising:
an extraction circuitry configured to extract preset keywords in a plurality of linguistic units from the converted character strings; and
a keyword conversion circuitry configured to convert the extracted keywords into standard comments associated with the extracted keywords in advance,
wherein the ultrasonic image storage further stores, in the plurality of linguistic units, the converted standard comments and recording times of the converted character strings in association with the ultrasonic image.

8. The apparatus of claim 7, wherein the optical image selection circuitry selects, in the plurality of linguistic units, an optical image obtained at substantially the same time as an imaging time of the ultrasonic image from the repeatedly obtained optical images,
the specifying circuitry specifies, in the plurality of linguistic units, a position of the probe on the selected body mark based on the selected optical image,
the display image generating circuitry generates, in the plurality of linguistic units, a display image concerning the selected body mark to which the probe mark indicating the specified position is added, and
the ultrasonic image storage further stores, in the plurality of linguistic units, the converted standard comment, the selected optical image, and the generated display image in association with the ultrasonic image.

9. The apparatus of claim 8, further comprising a combining circuitry configured to generate a composite display image, with a plurality of probe marks respectively indicating contact positions of the probe being added to the selected body mark, based on a plurality of display images concerning the plurality of linguistic units.

10. The apparatus of claim 9, wherein the ultrasonic image storage further stores the composite display image in association with the ultrasonic image.

11. The apparatus of claim 1, further comprising:
an input circuitry configured to input an instruction to store an ultrasonic image; and
an ultrasonic image storage configured to store the ultrasonic image based on an input to the input unit,
wherein the body mark selection circuitry selects a body mark based on the optical image obtained at substantially the same time as a time when the input unit has received the input.

12. The apparatus of claim 1, wherein the image analysis comprises contour extraction.

13. An ultrasonic diagnostic apparatus which generates an ultrasonic image concerning an object via a probe, the apparatus comprising:
a display image generating circuitry configured to generate a display image, with a probe mark indicating a position of the probe which corresponds to each of a plurality of contact positions of the probe on the object being added to a body mark representing an examination region of the object; and
a symptom information storage configured to store symptom information representing a symptom of the object at each of the plurality of contact positions in association with each of the plurality of contact positions.

14. The apparatus of claim 13, further comprising:
a recording circuitry configured to record speech;
a speech conversion circuitry configured to convert the recorded speech into character strings;
an extraction circuitry configured to extract preset keywords from the converted character strings in a plurality of linguistic units; and
a keyword conversion circuitry configured to convert the extracted keyword into a standard comment associated with the extracted keyword in advance,
wherein the symptom information storage stores, for each contact position of the probe, the body mark and the standard comment acquired at substantially the same time as the body mark in association with each other.

15. The apparatus of claim 13, further comprising:
an imaging circuitry configured to repeatedly obtain an optical image concerning the object;
a body mark storage configured to store data of a plurality of types of body marks;
a body mark selection circuitry configured to select a body mark corresponding to an examination region area of the object from the plurality of body marks by using the optical image; and
a combining circuitry configured to generate a composite display image, with a plurality of probe marks, each indicating a position of the probe at each of a plurality of contact positions of the probe, being added to the selected body mark,
wherein the display image generating circuitry adds the plurality of probe marks to the selected body mark upon position matching.

16. The apparatus of claim 15, wherein the symptom information storage registers the composite display image
in association with the ultrasonic image.
